(19) United States
(12) Patent Application Publication
(10) Pub. No.: US 2008/0069904 A1
(43) Pub. Date: Mar. 20, 2008

(54) Inhibition of angiogenesis through nitric oxide tachyphylaxis

(76) Inventors: Bryan T. Oronsky, Los Altos, CA (US); John W. Sliwa, Los Altos, CA (US); Herbert L. Berman, Los Altos Hills, CA (US); Carol A. Tosaya, Los Altos, CA (US); Neil Oronsky, Los Altos Hills, CA (US); Robin Martin, Bruxelles (BE)

Correspondence Address: David W. Collins - Intellectual Property Law, Suite 100, 512 E. Whitehouse Canyon Road, Green Valley, AZ, 85614, US

(21) Appl. No.: 11/827,487

(22) Filed: Jul. 11, 2007

Related U.S. Application Data
(60) Provisional application No. 60/831,651, filed on Jul. 18, 2006.

(51) Int. Cl. A61K 33/04; A61K 31/04; A61P 35/00

(52) U.S. Cl. 424/702; 514/742

(57) ABSTRACT

A method is provided for treating cancer in a mammalian subject by inhibiting angiogenesis through the administration to the subject of a therapeutically effective amount of a nitrate-containing compound and a therapeutically effective amount of an inorganic selenium-containing compound. Kits and systems are also disclosed for practicing the subject methods.

### CROSS-REFERENCE TO RELATED APPLICATION

The present application claims priority from provisional application Ser. No. 60/831,651, filed Jul. 18, 2006.

### BACKGROUND OF THE INVENTION

Tumor angiogenesis is the proliferation of a network of new vessels to establish an independent blood supply to the cancerous cells, ensuring the delivery of nutrients, oxygen and growth factors and the removal of waste products. Once a tumor gets beyond about 2 to 3 cubic millimeters in size, the availability of oxygen and nutrients via diffusion is severely limited. The malignant cells proliferate so rapidly that they are likely to outstrip their pre-existing blood supply and thus become dependent on angiogenesis. The ability of tumors to release and induce several angiogenic factors leads to the formation of new vessels which increase the delivery of oxygen and nutrients, thereby promoting continued tumor growth and ultimately metastasis, because the cells in the tumor can slough off into the vasculature and migrate to distinct anatomic sites. Failure of angiogenesis leads tumor “dormancy” and the interruption of an existing blood supply results in necrosis and apoptosis of tumor cells and tumor regression.

Tumor angiogenesis is thought to result from the secretion of “angiogenesis factors” by tumor cells; these include growth factors such as VEGF, cytokines, and also a number of small molecules. Evidence suggests that NO also promotes pathological angiogenesis. In solid tumors, inhibition of nitric oxide synthase or NOS, the enzyme responsible for the synthesis of NO, has an anti-angiogenic effect. (Roles of Nitric Oxide Synthase Inhibition and Vascular Endothelial Growth Factor Receptor-2 Inhibition on Vascular Morphology and Function in an In vivo Model of Pancreatic Cancer E. Ramsay Camp et al) In addition, tumor levels of NOS and cGMP are significantly higher in tumor tissue and metastases are more angiogenic than in non-metastatic tissue. (Nitric Oxide Donors Yamamoto and Bing Minireview.)

Drugs that have been used in an attempt to inhibit angiogenesis include: endostatin, thalidomide, AE-941 (Neovastat), bevacizumab (also known as Avastin, anti-VEGF, RhuMabVEGF) and celecoxib (Celebrex). All of these drugs are thought in part to exercise an anti-oncogenic effect by interfering with angiogenesis.

While significant advances have been made in dealing with tumor angiogenesis, no one drug has proven to be broadly effective for an extended time frame or have proven to be helpful in prevention of reoccurrence of cancer. The present invention addresses this need.

### SUMMARY OF THE INVENTION

The present invention provides a method of interfering with angiogenesis in a mammalian subject by administering to the subject a therapeutically effective amount of a nitrate-containing compound and a therapeutically effective amount of an inorganic selenium-containing compound. The present invention also provides kits and systems for practicing the subject methods.

A feature of the subject invention is a method of interfering with angiogenesis in a subject with cancer, comprising, administering to a subject with cancer a therapeutically effective amount of an organic nitrate-containing compound and a therapeutically effective amount of an inorganic selenium-containing (iSe) compound, wherein said administering decreases the incidence or interferes with the formation of new or existing tumor vessels. In some embodiments, the iSe compound is inorganic selenite. In some embodiments, the organic nitrate-containing compound is selected from the group consisting of glycerol trinitrate, isosorbide-dinitrate, isobutyl nitrate, isopentyl nitrate, and isosorbide-5-mononitrate.

Another feature of the subject invention is a method for interfering with angiogenesis in a subject with cancer by administering a subanginal dose of a organic nitrate-containing compound and a therapeutically effective amount of an inorganic selenium-containing (iSe) compound, wherein said administering interferes with angiogenesis in the subject. In some embodiments, the iSe compound is inorganic selenite. In some embodiments, the organic nitrate-containing compound is selected from the group consisting of glycerol trinitrate, isosorbide-dinitrate, isobutyl nitrate, isopentyl nitrate, and isosorbide-5-mononitrate.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic representing nitrate tolerance in response to continual organic nitrate-containing compound therapy.

FIG. 2 is a schematic representing the effect of organic nitrate-containing compound in generating NO and stimulating guanylate cyclase to cause vasodilatation.

### DETAILED DESCRIPTION OF THE INVENTION

### Definitions

The term “isolated compound” means a compound which has been substantially separated from, or enriched relative to, other compounds with which it occurs in nature. Isolated compounds are usually at least about 80%, more usually at least 90% pure, even more preferably at least 98% pure, most preferably at least about 99% pure, by weight. The present invention is meant to comprehend diastereomers as well as their racemic and resolved, enantiomerically pure forms and pharmaceutically acceptable salts thereof.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Of particular interest are human subjects.

“Mammal” means a member or members of any mammalian species, and includes, by way of example, canines; felines; equines; bovines; ovines; rodentia, etc. and primates, particularly humans. Non-human animal models, particularly mammals, e.g., primate, murine, lagomorpha, etc. may be used for experimental investigations.

“In combination with” as used herein refers to uses where, for example, the first compound is administered during the entire course of administration of the second compound; where the first compound is administered for a period of time that is overlapping with the administration of the second compound, e.g., where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds that may be administered in the same or different formulations, by the same of different routes, and in the same or different dosage form type.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound (e.g., phenylglycine-containing compound or sulfonamide containing compound) employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including, rectal, parenteral, intraperitoneal, intradermal, intracheal and the like.

As used herein, “pharmaceutically acceptable derivatives” of a subject compound includes salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

A “pharmaceutically acceptable salt” of a subject compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a subject compound means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a subject compound means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable enol ester” of a subject compound means an enol ester that is pharmaceutically acceptable and that possesses the desired pharmacological activity of the parent compound, and includes, but is not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a subject compound means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

“Pro-drugs” means any compound that releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of formula (I), and the like.

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not the environment of use.

By “substantially continuous” as used in, for example, the context of “substantially continuous subcutaneous infusion” or “substantially continuous delivery” is meant to refer to delivery of drug (e.g., nitrate-containing compound, an inorganic selenium-containing compound) in a manner that is substantially uninterrupted for a pre-selected period of drug delivery (other than a period associated with, for example, a bolus injection). Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Present Invention

The present invention provides a method of treating cancer in a mammalian subject by interfering with angiogenesis through the administration to the subject of a therapeutically effective amount of a nitrate-containing compound and a therapeutically effective amount of an inorganic selenium-containing compound. The present invention also provides kits and systems for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a subject” includes a plurality of such subjects and reference to “the compound” includes reference to one or more compounds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Overview of the Invention

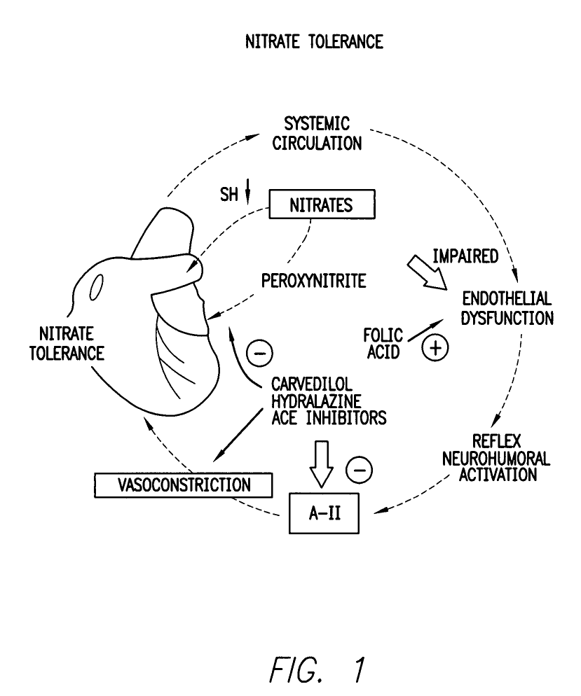

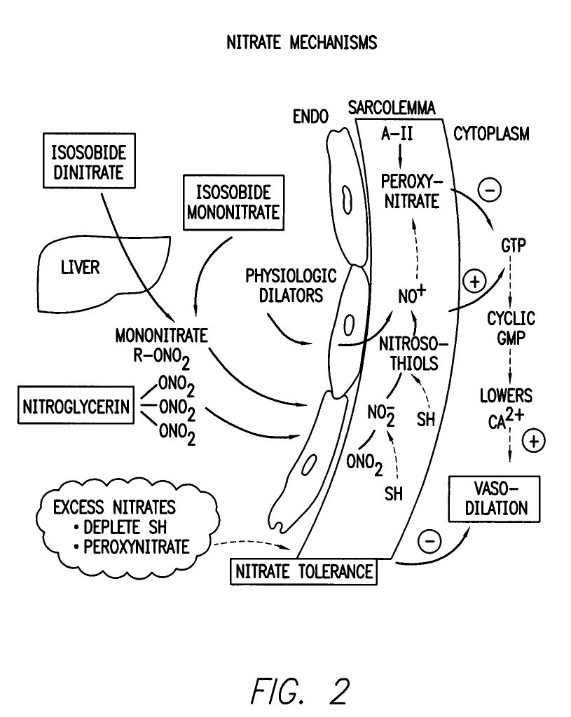

The present invention is based on the observation that while intermittent exposure to organic nitrate-containing compounds has a clinical effect, continuous exposure to organic nitrate-containing compounds results in tachyphylaxis. It is hypothesized that the mechanism of nitrate tolerance results in depletion of reduced sulfyhydryl groups and results in increased free radical production (see FIG. 1). Therefore, nitrate tolerance may be related to depletion of reduced sulfhydryl groups in vascular smooth muscle with resultant reduction in nitric oxide production, adenylate cyclase activation and cyclic guanosine monophosphate (GMP) production. Accordingly, therapy with organic nitrogen-containing compounds, such as nitroglycerin, and possibly other nitric-oxide donors, increases superoxide production, increases superoxide production, thereby causing endothelial dysfunction and heightened vascular responsiveness to vasoconstrictors such as angiotensin II (seen in the figures), which thus impairs both responses to nitrates and agents that stimulate NO release from the endothelium (see FIG. 2).

The inorganic form of selenium, selenite (SeO₃⁻²) undergoes thiol-dependent reduction to produce selenide (H₂Se) which supplies selenium for the synthesis of selenoproteins. At lower concentrations, the major effects of selenite are related to its role as a micronutrient. However, at higher concentrations, selenite can result in free radical generation. In its reaction with selenite, reduced glutathione (GSH) is oxidized to its oxidized form (GSSG). The GSH-dependent reduction of selenite and the further oxidative metabolism of the resulting selenide can therefore produce superoxide anions and induce oxidative stress. Therefore, it is hypothesized that selenite may augment the effect of continual nitrate therapy on the induction of nitrate tolerance through resulting depletion of reduced sulfhydryl groups and increased free radical production.

The present invention is described in greater detail below.

Methods

As noted above, the present invention provides methods for preventing and/or treating cancer by inhibiting angiogenesis through the administration to a subject in a continuous fashion an effective amount of an organic nitrate-containing compound and a therapeutically effective amount of an inorganic selenium-containing (iSe) compound.

Typically, a tumor secretes angiogenic factors in response to hypoxia, which occurs when the existing blood supply is outstripped by the rapid growth of the cancer cells.

Subjects suitable for treatment include individuals that have, or are at risk of, any kind of cancer since all tumors depend angiogenesis.

Organic nitrate-containing compounds suitable for use in the invention can be provided in a variety of forms. Examples of suitable organic nitrate-containing compounds include, but are note limited to, glycerol trinitrate (nitroglycerin), isosorbide-dinitrate, isosorbide-5-mononitrate, isobutyl nitrate, and isopentyl nitrate. In representative embodiments the organic nitrate-containing compound is glycerol trinitrate (nitroglycerin), both because of its ready availability in a variety of forms; pill, patch, ointment, cream, spray, inhaler, etc., and because its pharmacology is so well known. The many Nitroglycerine equivalents and substitutes, such as p.o. clonidine, Dynacirc (isradipine), hydrazine, or long acting nifedipine and others known to the art, can be used to replace or to supplement Nitroglycerine.

Inorganic selenium-containing compound suitable for use in the invention can be provided in a variety of forms. It is noted that selenium may be present in elemental form or as inorganic or organic selenium compounds. It is also noted that selenium occurs in a number varying valence forms. For example, selenium compounds occur in which the selenium has a +4 valance or a +6 valence, as the selenite and selenate ions, respectively. Preferably, the selenium-containing compound is an inorganic selenium-containing compound, referred to herein as an “iSe compound”. It is to be understood, however, that the particular inorganic forms of selenium compounds set forth herein are not to be considered limitative.

Among the inorganic selenite and selenate forms, of interest for use in the compositions of this invention are the water-soluble alkali metal salts thereof, and particularly, the sodium and potassium salts, that is, sodium and potassium selenite and selenate. Of particular interest for use in the compositions of this invention are the water-soluble alkali metal salts of selenite, and particularly, the sodium and potassium salts, that is, sodium selenite and potassium selenite.

Pharmaceutical Preparations

The subject compounds can be incorporated into a variety of formulations for therapeutic administration by a variety of routes. More particularly, the organic nitrate-containing compound and/or inorganic selenium-containing compound can be formulated into pharmaceutical compositions by combination with appropriate, pharmaceutically acceptable carriers, diluents, excipients and/or adjuvants, and may be formulated into preparations in solid, semi-solid, liquid or gaseous forms, such as tablets, capsules, powders, granules, ointments, solutions, suppositories, injections, inhalants and aerosols. The organic nitrate-containing compounds and inorganic selenium-containing compound can be formulated together and administered to the subject at the same time or can be formulated separately and administered to the subject at the same time or at a different time.

Pharmaceutically acceptable excipients usable with the invention, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

Dosage Forms

In pharmaceutical dosage forms, the organic nitrate-containing compound and the inorganic selenium-containing compound may be administered in the form of a pharmaceutically acceptable salt, or may also be used alone or in appropriate association, as well as in combination, with other pharmaceutically active compounds. The following methods and excipients are merely exemplary and are in no way limiting.

The subject compounds can be administered to a host using any available conventional methods and routes suitable for delivery of conventional drugs, including systemic or localized routes. In general, routes of administration contemplated by the invention include, but are not necessarily limited to, enteral or parenteral routes, such as intrapulmonary or intranasal delivery.

Conventional and pharmaceutically acceptable routes of administration include intramuscular, intrathecal, intratracheal, subcutaneous, intradermal, intravenous, transdermal, rectal, nasal, oral and other parenteral routes of administration. Routes of administration may be combined, if desired, or adjusted depending upon the agent and/or the desired effect. For example, the nitrate-containing compound may be administered by a topical route (e.g., transdermal route) and the inorganic selenium-containing compound may be administered by a different route, such as oral route or intravenous route. The compositions can be administered in a single dose or in multiple doses.

In one embodiment, the organic nitrate-containing compound is administer by topical administration (e.g., by transdermal administration). Topical formulations can be in the form of a transdermal patch, ointment, paste, lotion, cream, gel, and the like. Topical formulations may include one or more of a penetrating agent, thickener, diluent, emulsifier, dispersing aid, or binder. Where the compound is formulated for transdermal delivery, the compound may be formulated with or for use with a penetration enhancer. Penetration enhancers, which include chemical penetration enhancers and physical penetration enhancers, facilitate delivery of the compound through the skin, and may also be referred to as “permeation enhancers” interchangeably. Physical penetration enhancers include, for example, electrophoretic techniques such as iontophoresis, use of ultrasound (or “phonophoresis”), and the like. Chemical penetration enhancers are agents administered either prior to, with, or immediately following compound administration, which increase the permeability of the skin, particularly the stratum corneum, to provide for enhanced penetration of the drug through the skin.

Compounds that have been used to enhance skin permeability include: the sulfoxides dimethylsulfoxide (DMSO) and decylmethylsulfoxide (C10 MSO); ethers such as diethylene glycol monoethyl ether, dekaoxyethylene-oleylether, and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin; the 1-substituted azacycloheptan-2-ones, particularly 1-n-dodecylcyclazacycloheptan-2-one; alcohols such as ethanol, propanol, octanol, benzyl alcohol, and the like; petrolatums, such as petroleum jelly (petrolatum), mineral oil (liquid petrolatum), and the like; fatty acids such as C₈-C₂₂ and other fatty acids (e.g., isostearic acid, octanoic acid, oleic acid, lauric acid, valeric acid); C₈-C₂₂ fatty alcohols (e.g., oleyl alcohol, lauryl alcohol); lower alkyl esters of C₈-C₂₂ fatty acids and other fatty acids (e.g., ethyl oleate, isopropyl myristate, butyl stearate, methyl laurate, isopropyl myristate, isopropyl palmitate, methylpropionate, ethyl oleate); monoglycerides of C₈-C₂₂ fatty acids (e.g., glyceryl monolaurate); tetrahydrofurfuryl alcohol polyethylene glycol ether; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; di-lower alkyl esters of C₆-C₈ diacids (e.g., diisopropyl adipate); ethyl acetate; acetoacetic ester; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, N-alkylpyrrolidone, e.g., 1-methyl-2-pyrrolidone; ethanol amine, diethanol amine and triethanolamine; terpenes; alkanones, and organic acids, particularly salicylic acid and salicylates, citric acid and succinic acid. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneruas et al., J Pharm Pharmacol 2002; 54(4):499-508; Karande et al., Pharm Res 2002; 19(5):655-60; Vaddi et al., J Pharm Sci 2002 July; 91(7):1639-51; Ventura et al., J Drug Target 2001; 9(5):379-93; Shokri et al., Int J Pharm 2001; 228(1-2):99-107; Suzuki et al., Biol Pharm Bull 2001; 24(6):698-700; Alberti et al., J Control Release 2001; 71(3):319-27; Goldstein et al., Urology 2001; 57(2):301-5; Kiijavainen et al., Eur J Pharm Sci 2000; 10(2):97-102; and Tenjarla et al., Int J Pharm 1999; 192(2):147-58.

Where the compound is formulated with a chemical penetration enhancer, the penetration enhancer is selected for compatibility with the compound, and is present in an amount sufficient to facilitate delivery of the compound through skin of a subject, e.g., for delivery of the compound to the systemic circulation.

In one embodiment, one or both of the compounds are provided in a drug delivery patch, e.g., a transmucosal or transdermal patch, and can be formulated with a penetration enhancer. The patch generally includes a backing layer, which is impermeable to the compound and other formulation components, a matrix in contact with one side of the backing layer, which matrix provides for sustained release, which may be controlled release, of the compound, and an adhesive layer, which is on the same side of the backing layer as the matrix. The matrix can be selected as is suitable for the route of administration, and can be, for example, and can be a polymeric or hydrogel matrix.

For oral preparations, the subject compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Parenteral routes of administration other than inhalation administration include, but are not necessarily limited to, subcutaneous, intramuscular, intrathecal, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The subject compounds can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agent can also be delivered to the subject by enteral administration. Enteral routes of administration include, but are not necessarily limited to, oral and rectal (e.g., using a suppository) delivery.

Furthermore, the subject compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Dosages

Depending on the subject and the administration route, the organic nitrate-containing compound and the inorganic selenium-containing compound may each be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

In some embodiments, the dosage of organic nitrate-containing compound administered to the subject is selected in order to provide for a subanginal dose of a organic nitrate-containing compound, including about 0.1 mg/hr to about 0.3 mg/hr of organic nitrate-containing compound, including about 0.11 mg/hr to about 0.29 mg/hr, about 0.12 mg/hr to about 0.28 mg/hr, about 0.13 mg/hr to about 0.27 mg/hr, about 0.14 mg/hr to about 0.26 mg/hr, about 0.15 mg/hr to about 0.25 mg/hr, about 0.16 mg/hr to about 0.24 mg/hr, about 0.17 mg/hr to about 0.23 mg/hr, about 0.18 mg/hr to about 0.22 mg/hr, about 0.19 mg/hr to about 0.21 mg/hr, about 0.20 mg/hr of organic nitrate-containing compound.

In representative embodiments, the organic nitrate-containing compound will be nitroglycerin and the administration route will be topical, e.g., transdermal administration. An exemplary transdermal delivery system for nitroglycerin is the NITRO-DUR™ patch, such as the 10 cm² NITRO-DUR™ patch comprising 40 mg of nitroglycerin or the 2.5 cm² NITRO-DUR™ patch comprising 10 mg of nitroglycerin. The 10 cm² NITRO-DUR™ patch can be cut in half to provide two 5 cm² NITRO-DUR™ patches each comprising 20 mg of nitroglycerin. In other embodiments, the nitroglycerin is provided in a 2% ointment for topical application as cream or rub. The ointment can be applied to a 0.5 inch² area once a day to provide for delivery of 20 mg of nitroglycerin. The ointment can be applied to a 0.25 inch² area once a day to provide for delivery of 10 mg of nitroglycerin.

In some embodiments, the dosage of inorganic selenium-containing compound administered to the subject is selected in order to provide for a dose of a organic nitrate-containing compound, including about 1 μM/kg to about 20 μM/kg, about 2 μM/kg to about 19 μM/kg, about 3 μM/kg to about 18 μM/kg, about 4 μM/kg to about 17 μM/kg, about 5 μM/kg to about 16 μM/kg, about 6 μM/kg to about 15 μM/kg, about 7 μM/kg to about 14 μM/kg, about 8 μM M/kg to about 13 μM/kg, about 9 μM/kg to about 12 μM/kg, about 10 μM/kg to about 11 μM/kg, and the like.

Typically, the duration of the administration of organic nitrate-containing compound and inorganic selenium-containing compound will be continual and range from about 1 week to about 6 months or more, including about 22 weeks, about 20 weeks, about 18, weeks, about 16 weeks, about 15 weeks, about 14 weeks, about 13 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, etc. In some embodiments, the duration of the administration of the subject compounds may be more, such as more than about 6, months, including about 7 moths or more, about 8 months or more, about 9 months or more, about 10 months or more, about 1 year or more, about 18 months or more, etc.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of the subject composition to provide for a desired effect in a subject animal.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the invention. Similarly, unit dosage forms for injection or intravenous administration may comprise the compound (s) in a composition as a solution in sterile water, normal saline or another pharmaceutically acceptable carrier.

Combination Therapy

For use in the subject methods, the subject compounds may be formulated with or otherwise administered in combination with other pharmaceutically active agents, including other compounds useful for treating migraines. The subject compounds may be used to provide an increase in the effectiveness of another chemical, such as a pharmaceutical, or a decrease in the amount of another chemical, such as a pharmaceutical, that is necessary to produce the desired biological effect.

For example, the subject methods can be combined with other anti-angiogenic inhibitors, such as thalidomide, endostatin, AE-941 (Neovastat), bevacizumab, anti-VEGF and celecoxib, since the addition of these agents may have synergistic effects.

The compounds described herein for use in combination therapy with subject compounds may be administered by the same route of administration (e.g., intrapulmonary, oral, enteral, etc.) that the compounds are administered. In the alternative, the compounds for use in combination therapy with the subject compounds may be administered by a different route of administration that the compounds are administered.

Kits and Systems

Kits with unit doses of organic nitrate-containing compound and the inorganic selenium-containing compound are provided. In such kits, in addition to the sterile containers containing the unit doses will be an informational package insert describing the use and attendant benefits of the subject compounds in treating the pathological condition of interest. Preferred compounds and unit doses are those described herein above. In some embodiments, the organic nitrate-containing compound is provided for topic administration, such as, for example, a transdermal patch. In some embodiments, the selenium-containing compound is provided for oral administration.

### EXAMPLES

The following example is put forth so as to provide those of ordinary skill in the art with a complete disclosure and description of how to make and use the present invention, and is not intended to limit the scope of what the inventors regard as their invention nor are they intended to represent that the experiment below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example

Organic nitrate-containing compound and the inorganic selenium-containing compound are administered to subjects. Subjects suitable for treatment include individuals that have, or are at risk of, cancer. The organic nitrate-containing compound is nitroglycerin and the administration route is by transdermal administration in a continual subanginal dose of about 20 mg/hr. The NITRO-DUR™ patch is used, such as the 10 cm² NITRO-DUR™ patch comprising 40 mg of nitroglycerin. The 10 cm² NITRO-DUR™ patch is cut in half to provide two 5 cm² NITRO-DUR™ patches, each comprising 20 mg of nitroglycerin, and one 5 cm² NITRO-DUR™ patch is administered at a time to each subject. The dosage of inorganic selenium-containing compound administered to the subject is about 10 μM/kg. The administration of subject compounds is continued for a period of about 1 month to about 3 months.

The effectiveness of the administration of organic nitrate-containing compound and the inorganic selenium-containing compound on the treatment or prevention of angiogenesis is assessed by measuring tumor growth and vessel counts.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating cancer by inhibiting angiogenesis, comprising:
administering to a subject suffering from cancer a therapeutically effective amount of an organic nitrate-containing compound and a therapeutically effective amount of an inorganic selenium-containing (iSe) compound,
wherein said administering interferes with angiogenesis in the subject.

2. The method of claim 1, wherein the iSe compound is inorganic selenite.

3. The method of claim 1, wherein the organic nitrate-containing compound is selected from the group consisting of glycerol trinitrate, isosorbide-dinitrate, isobutyl nitrate, isopentyl nitrate, and isosorbide-5-mononitrate.

4. A method for preventing recurrence of chronic migraine episodes, comprising:
administering to a subject a subanginal dose of a organic nitrate-containing compound and a therapeutically effective amount of an inorganic selenium-containing (iSe) compound,
wherein said administering prevents recurrence of chronic migraine episodes in the subject.

5. The method of claim 1, wherein the iSe compound is inorganic selenite.

6. The method of claim 1, wherein the organic nitrate-containing compound is selected from the group consisting of glycerol trinitrate, isosorbide-dinitrate, isobutyl nitrate, isopentyl nitrate, and isosorbide-5-mononitrate.

7. A method of treating cancer by inhibiting angiogenesis, comprising:
administering to a subject suffering from cancer a therapeutically effective amount of an organic nitrate-containing compound and a therapeutically effective amount of an inorganic selenium-containing (iSe) compound, in combination with another anti-angiogenic agent such as thalidomide, endostatin, AE-941 (Neovastat), bevacizumab, anti-VEGF and celecoxib,
wherein said administering inhibits angiogenesis in the subject.

8. A method of treating cancer by inhibiting angiogenesis, comprising:
administering to a subject suffering from cancer a therapeutically effective amount of an organic nitrate-containing compound and a therapeutically effective amount of an inorganic selenium-containing (iSe) compound, in combination with other anti-cancer agents such as but not limited to alkylating agents, antimetabolites, anthracylines and anthracenediones, bleomycin, vinca alkaloids, taxanes, epipodophyllotoxins, camptothecin derivatives, cisplatin and carboplatin, hydroxyurea, and L-Asparaginase.

9. A kit for treating cancer by inhibiting angiogenesis, said kit containing unit doses of a therapeutically effective amount of an organic nitrate-containing compound and a therapeutically effective amount of an inorganic selenium-containing (iSe) compound.
